(19) United States
(12) Patent Application Publication
(10) Pub. No.: US 2022/0161022 A1
(43) Pub. Date: May 26, 2022

(54) A VASCULAR COUPLING DEVICE

(71) Applicant: SCANDINAVIAN REAL HEART AB, Västerås (SE)

(72) Inventor: Azad Najar, Västerås (SE)

(73) Assignee: SCANDINAVIAN REAL HEART AB, Västerås (SE)

(21) Appl. No.: 17/601,482

(22) PCT Filed: Apr. 3, 2020
(86) PCT No.: PCT/EP2020/059644

§ 371 Date: Oct. 5, 2021

(30) Foreign Application Priority Data
Apr. 5, 2019 (SE) 1950431-5
Aug. 26, 2019 (SE) 1950977-7

(51) Int. Cl. A61M 60/859; A61M 60/196; A61M 60/258; A61M 60/424; A61M 60/50

(52) U.S. Cl. CPC A61M 60/859; A61M 60/196; A61M 60/50; A61M 60/424; A61M 60/258

(57) ABSTRACT

A vascular coupling device (10), comprising a first and a second coupling element (21, 22) wherein each one of said first and second coupling elements (21, 22) has an external surface (23′, 23″) facing an external side, a coupling surface (25′, 25″) facing a coupling side, a central opening (27′, 27″), and a first and a second tubular connecting element (31, 32). Each one of said first and second tubular connecting elements (31, 32) is arranged in a corresponding central opening (27′, 27″) of the first and second coupling elements (21, 22) respectively, and with second open ends (37, 38) protruding through said central openings (27′, 27″) on said external side of each of said first and second coupling elements (21, 22). The first and second coupling elements (21, 22) being removably connected to each other into a locked configuration, or disconnected from each other into an unlocked configuration by means of a first and second locking structure (41, 42) being arranged on a centerline A and opposite to each other on an outer perimeter of said vascular coupling device (10). The vascular device further comprises a fail-safe arrangement comprising first and second cut-in portions (91, 92) arranged on said first coupling element (21) configured to receive first and second projecting elements (93, 94) arranged on said second coupling element (22), thereby preventing erroneous connection of said first and second coupling elements (21, 22) to each other.

### TECHNICAL FIELD

The present invention relates to a vascular coupling device for connecting any heart prosthesis such as a total artificial heart (TAH) to the vascular system of a subject in need of a circulatory support system. A method for connecting a TAH to the vascular system of a subject is also disclosed.

### BACKGROUND OF THE INVENTION

The main function of the heart in the human body is to circulate blood through the blood vessels in order to transport oxygen, nutrition, and waste products to and from body cells. Many diseases may affect the heart such as myocardial infarction, hypertension, valve insufficiency and various heart muscle diseases. The end result of such diseases may be heart failure which means that the heart has lost its ability to pump enough blood to the lungs and body tissues.

The symptoms of heart failure are shortness of breath, edema and fatigue. The only treatment option available for a patient suffering from advanced heart failure is heart transplantation. However, due to a lack of sufficient number of donor hearts, the majority of advanced heart failure patients die while waiting for a heart transplant operation.

For this reason many efforts have been made during the last 50 years to develop a mechanical heart which can replace a diseased heart entirely. Until now only a few Total Artificial Hearts (TAH) i.e. mechanical hearts/heart prostheses have been developed which have the capacity to completely replace the diseased heart.

WO2016/020219 discloses a four-chambered TAH which is designed as a human heart. This TAH comprises a first and a second artificial heart pump corresponding to the left and right heart of the natural heart. Each artificial pump comprises an inlet and an outlet channel and a valve cylinder which is divided into two chambers by means of a moving plane comprising a one-way valve which corresponds to the atrioventricular (AV) plane in a natural heart. Pump actuating means are configured to apply a movement to the valve cylinders in an upward and downward direction in response to control signals from a control unit such that when the valve cylinders move in an upward direction inside the blood pump housing device, the valves provided in the valve planes are in an open position allowing a flow of blood through the inlet channel into the artificial atrium, and thereafter into the artificial ventricle. When the valve cylinders move in a downward direction the valves are in the closed position and blood is ejected from the artificial ventricle and exit therefrom through outlet channels.

The TAH is enclosed in a casing which protects the surrounding tissue from moving parts and prevents entry of body fluids into the TAH. When implanted in a subject the diseased natural heart of the subject is removed and thereafter the circulatory system of the subject is connected to the inlets and outlets of the TAH. The coupling device connecting the vascular system with the inlets and outlets of the TAH has to be absolutely leak-proof as well as easy to connect and disconnect from the TAH. Furthermore, the vascular coupling device must have the capacity to elastically accommodate any body movements of the subject without damaging the vascular system of the patient, or disconnecting it from the TAH.

### SUMMARY OF THE INVENTION

The above objects may be achieved with a vascular coupling device in accordance with independent claim 1, and a TAH having inlet and outlet channels connected to the vascular coupling device according to independent claim 18. Further embodiments are set out in the dependent claims, the description and in the drawings.

The vascular coupling device as disclosed herein will provide an absolutely leak-proof coupling between the vascular system of the patient and the inlets and outlets of a TAH. The vascular coupling device is easily connected to the circulatory system of the subject, i.e. both the systemic circulation circuit and the pulmonary circulation circuit as well as to the TAH in a leak-proof and flexible manner.

As set out herein, there is provided a vascular coupling device comprising a first and a second coupling element wherein each one of the first and second coupling elements has an external surface facing an external side, a coupling surface facing a coupling side, and a central opening.

The vascular coupling device further comprises a first and a second tubular connecting element, wherein each one of the first and second tubular connecting elements has a first open end and a second open end. The first open ends are provided with flanges. Each one of the first and second tubular connecting elements is arranged in a corresponding central opening of the first and second coupling elements respectively, while the second open ends of the first and second tubular connecting elements protrude through the central openings on the external side of each of the first and second coupling elements.

The first and second coupling elements are removably connected to each other into a locked configuration, or disconnected from each other into an unlocked configuration by means of a first and second locking structure. The first and second locking structures are arranged on a centerline A and opposite to each other and on an outer perimeter of the vascular coupling device.

The vascular device further comprises a fail-safe arrangement comprising first and second cut-in portions arranged on said first coupling element configured to receive first and second projecting elements arranged on said second coupling element, thereby preventing erroneous connection of said first and second coupling elements to each other.

The inner perimeters of the central openings are advantageously provided with grooves arranged facing the coupling sides of the coupling elements, and the flanges of the first open ends of the first and second tubular connecting elements are advantageously provided with upwardly turned lips. Advantageously the flanges with upwardly turned lips rest in the grooves of the central opening to provide a secure connection of the tubular connecting element to the first and second coupling elements.

Each one of the first and second locking structure comprises a female locking part arranged on the first coupling element, a male locking part arranged on the second coupling element and a locking element configured to join the female locking part with the male locking part into a locked configuration wherein the coupling surfaces of the first and second coupling elements are brought into contact such that the first open ends of the first and second tubular connecting elements are brought into a sealing connection.

Each one of the female locking parts comprises a first and a second lug having a gap therebetween, and the first and second lugs are arranged on opposite sides B and C of the centerline A. The first lug arranged on the side B has a first bore and the second lug arranged on the side C has a second bore, wherein the second bore is provided with an inner screw thread.

Each one of the male locking parts is arranged on the centerline A and is configured to be received into the gap between the first and second lugs of the female locking parts.

Each one of the locking elements has a first end provided with an outer screw thread, a second end provided with a cone shaped head, and a mid-section arranged between the first and second ends. The locking elements are configured to be received into the first and second bores of the female locking part.

Both of the locking elements are arranged to be entered into the first bores from a first side B and perpendicular to the centerline A, cross the centerline A and thereafter be received into the second bores provided with screw threads and arranged on a second side C of the centerline A, and opposite to the first side B.

Each one of the male locking parts comprises an eye provided with an open cleft facing the coupling side of the second coupling element.

The mid-sections of the locking elements are configured to be received into the open clefts provided on the eyes of the male locking parts when the male locking parts are arranged in the gaps between the first and second lugs of the female locking parts.

First and second recesses are arranged around inner perimeters of the eyes and on same side of said centerline A of the male locking parts. The first and second recesses are configured to receive the cone-shaped heads of the locking elements.

When the vascular device is in a locked configuration, the outer screw threads of the locking elements are received into and joined with the inner screw threads provided in the second bores of the female locking parts, and the cone shaped heads of the locking elements are received into the recesses provided on the eyes arranged on the male locking parts.

The first and second projecting elements are arranged on an outer perimeter of the second coupling element on the same side of the centerline A.

The first and second cut-in portions are arranged on an outer perimeter and on the same side of the centerline A on the first coupling element.

An encasing sac is attached to the external surfaces of the coupling device and receives and encloses the artificial heart pump to protect the pump from tissue ingrowth.

A first and second receiving means are arranged on the first and second coupling elements respectively, both first and second receiving means being arranged on the same side of the center line A and are adapted to facilitate manipulation of said vascular coupling device during implantation of the vascular coupling device.

The vascular coupling device as described herein is advantageously connected to one or more of the first and second inlet and outlet channels of a total artificial heart.

### BRIEF DESCRIPTION OF THE DRAWINGS

FIGS. 1A-D disclose the vascular coupling device A) in an exploded configuration, B) in an unlocked configuration, C) in a connected configuration and D) in a connected and locked configuration.

FIGS. 2A and B disclose the first and second coupling elements as viewed from the coupling sides.

FIGS. 3A and B are views of the first and second tubular connecting elements.

FIGS. 4A-C are cross-sectional views of a vascular coupling device in A) in an exploded configuration, B) in an unlocked configuration and C) in a connected configuration.

FIGS. 5A-d are views of the vascular coupling device when connected to a total artificial heart.

FIGS. 6A-D are views of A) the locking element, B) the locking element in a vascular coupling device when in an unlocked configuration, C) the locking element in a vascular coupling device when in a connected configuration and D) the locking in a vascular coupling device when in a locked configuration.

FIGS. 7A and B are views of the vascular coupling device when connected to a total artificial heart.

FIG. 8 is a view of an encasing sac enclosing the total artificial heart.

FIG. 9 discloses the receiving means adapted for receiving holding means for holding and manipulating the vascular coupling device during the implantation process.

FIGS. 10A and 10B disclose the vascular coupling device wherein holding means for holding and manipulating the vascular coupling device during the implantation process are attached.

### DETAILED DESCRIPTION

In the following detailed description, reference is made to the accompanying set of drawings that form a part of the description hereof and in which several specific embodiments are shown by way of illustration. It is to be understood that other embodiments are contemplated and may be made without departing from the scope of the present invention. The following detailed description, therefore, is not to be taken in a limiting sense.

The terms “a”, “an”, and “the” include plural referents unless the content clearly dictates otherwise. The term “or” is generally employed in its sense including “and/or” unless the content clearly dictates otherwise.

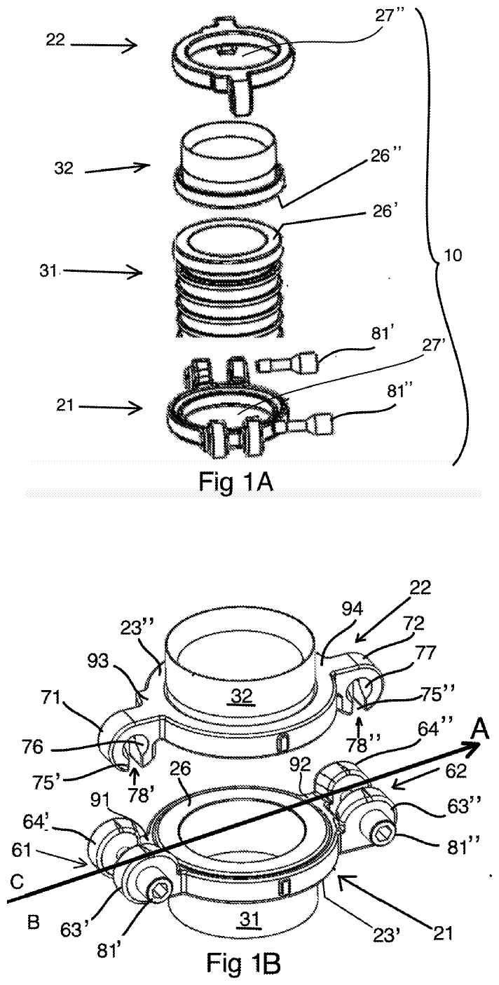

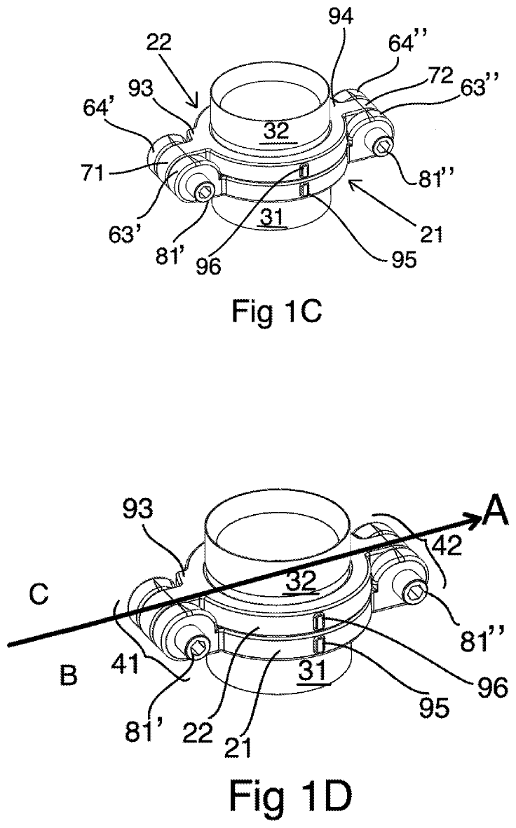

FIGS. 1-4 disclose a vascular coupling device 10 for connecting an artificial heart pump of a TAH to the vascular system of a subject. The vascular coupling device 10 is easily connected and/or disconnected to the artificial heart pump and forms a leak-proof and safe conduit between the patient's vascular system and the artificial heart pump.

The vascular coupling device 10 comprises a first and a second coupling element 21, 22, a first and second tubular connecting element 31, 32 and a first and second locking structure 41, 42, arranged to reversibly connect or disconnect the first and second coupling elements 21, 22 into a locked or unlocked configuration (see FIGS. 1A-D). A centerline A for the vascular coupling device 10 extends through the pair of first and second locking structures 41, 42 which are arranged on the centerline A and opposite to each other on an outer perimeter of said vascular coupling device 10 (see FIG. 1D).

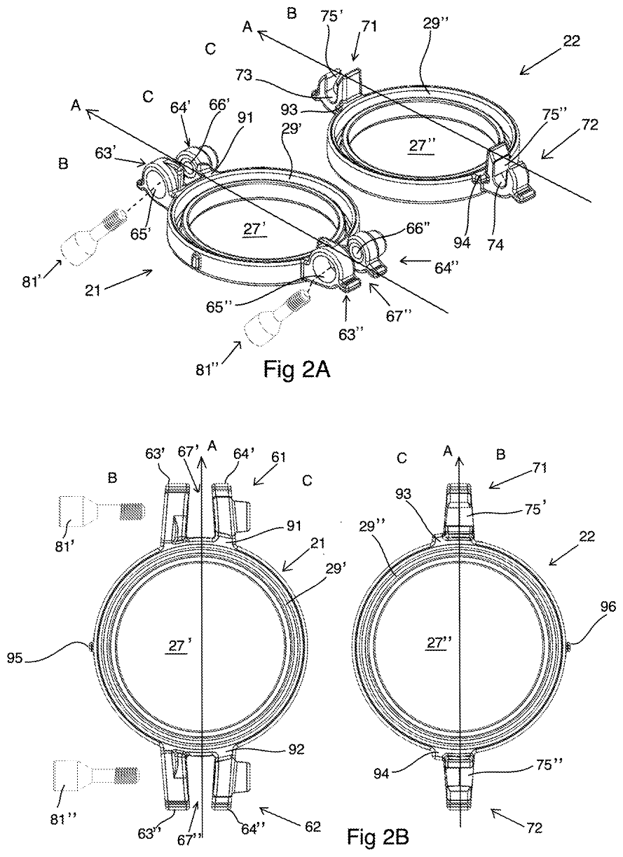

Each one of the first and second coupling elements 21, 22 are provided with a central opening 27′, 27″ (see FIGS. 2A-B). The central openings 27′, 27″ may have any geometric outline such as circular, oval, quadratic or rectangular, extending in the same plane as the centerline A. In the embodiments shown in the figures herein, the central openings 27′, 27″ are circular. In embodiments wherein the central openings 27′, 27″ are circular the inner diameter of the openings 27′, 27″ may have a length of 10-50 mm, preferably 20-40 mm, more preferably 25-35 mm. The first and second coupling elements 21, 22 generally have the same outline as the central openings 27′, 27″. This means that if the central openings 27′, 27″ have a circular configuration, also the first and second coupling elements 21, 22 will have a generally circular outline. The first and second tubular connecting elements 31, 32 are removably fitted inside the circular openings 27′, 27″ of the first and second coupling elements 21, 22 and they can be freely rotated therein. The rotational capability of the tubular connecting elements 31, 32 in relation to the coupling elements 21, 22 is a great advantage during the implantation process since the surgeon may then adjust the fitting of the vascular coupling device 10 to the patient as well as the TAH in an optimal way.

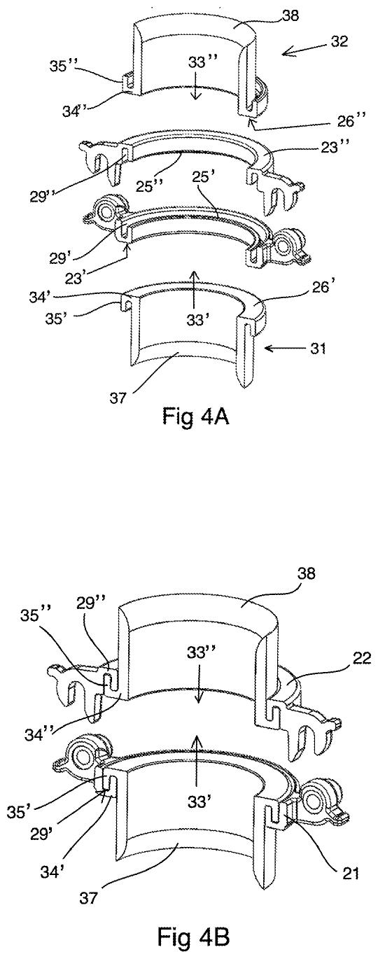

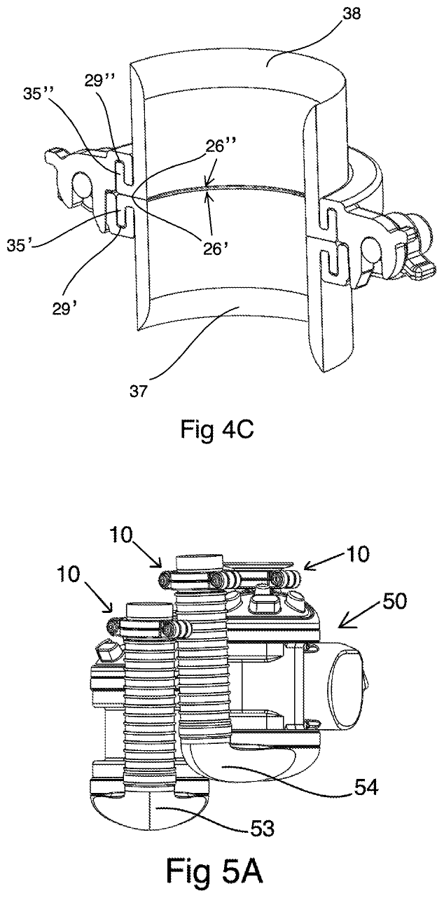

Each one of the coupling elements 21, 22 has an external side 23′, 23″ arranged opposite to a coupling side (see FIGS. 1B and 4A). The external sides 23′, 23″ of the first and second coupling elements 21, 22 face the exterior of the vascular device 10, and coupling surfaces 25′, 25″ (see FIG. 4A) provided on the coupling sides are arranged to face each other when the first and second coupling elements 21, 22 of the vascular coupling device are connected into a locked configuration.

The inner perimeters of the central openings 27′, 27″ are provided with grooves 29′, 29″ facing towards the coupling sides 25′, 25″ of the coupling elements 21, 22 (see FIGS. 2A-B). Each one of the first and second coupling elements 21, 22 have a flat configuration of 2-8 mm, 3-7 mm in height.

The first and second coupling elements 21, 22 are advantageously made from a biocompatible material such as stainless steel, titanium and any other stiff biocompatible materials.

Each one of the central openings 27′, 27″ provided in the first and second coupling elements 21, 22 are arranged to receive a tubular connecting element 31, 32 therein. The tubular connecting elements 31, 32 generally have a tubular configuration with a geometric cross-section that corresponds to the geometric outline of the central openings 27′, 27″ of the coupling elements 21, 22. The tubular connecting elements 31, 32 are provided with a first open end 33′, 33″ and a second open end 37, 38 (see FIG. 4A-C). The tubular connecting elements 31, 32 are made from a bio-compatible material.

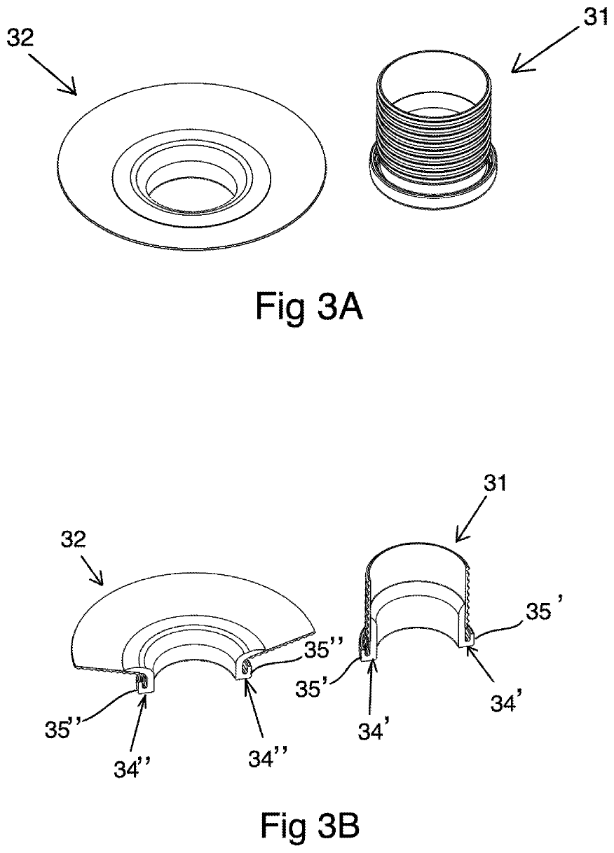

Advantageously, the first open ends 33′, 33″ of both the first and second tubular connecting elements 31, 32 are provided with a flange 34′, 34″ with an downwardly/upwardly turned lip 35′, 35″ (see FIGS. 3B and 4A-C). The downwardly/upwardly turned lips 35′, 35″ are arranged to be received and rest in the grooves 29′, 29″ provided around the inner parameters of the central openings 27′, 27″ on the coupling sides of the first and second coupling elements 21, 22. When the first and second tubular connecting elements 31, 32 are fitted into the central openings 27′, 27″ of the first and second coupling elements 21, 22, the flanges 34′, 34″ will be flush with the surfaces of the coupling sides forming a coupling surface 26′, 26″ for connecting the first open ends 33′, 33″ of the first and second tubular connecting elements 21, 22 in a leak-proof manner (see FIG. 4C).

The second open ends 37, 38 of each one of the first and second tubular connecting elements 31, 32 protrude through the central openings 27′, 27″ towards the exterior sides of each of the first and second coupling elements 21, 22 (see FIGS. 1B-D and 4B-C).

In order to facilitate the fitting of the tubular connecting elements 31, 32 to the first and second coupling elements 21, 22, the material of the flanges 34′, 34″ is advantageously of a pliable and resilient character that immediately resumes its original shape after having been fitted into or removed from the central openings 27′, 27″. The material of the flanges 34′, 34″ and the tubular connecting elements 31, 32 may be chosen from materials of the group consisting of polyethylene, polyamide, polymethylmethacrylate, polytetrafluroethylene, polyurethane, and silicones such as dimethyl siloxane, polydimethylsiloxane, and decamethyl cyclopentasiloxane, or combinations thereof.

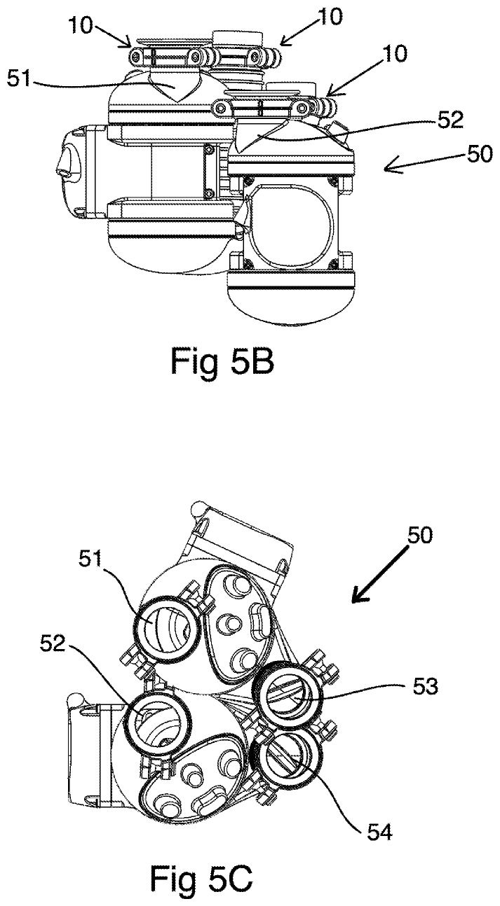

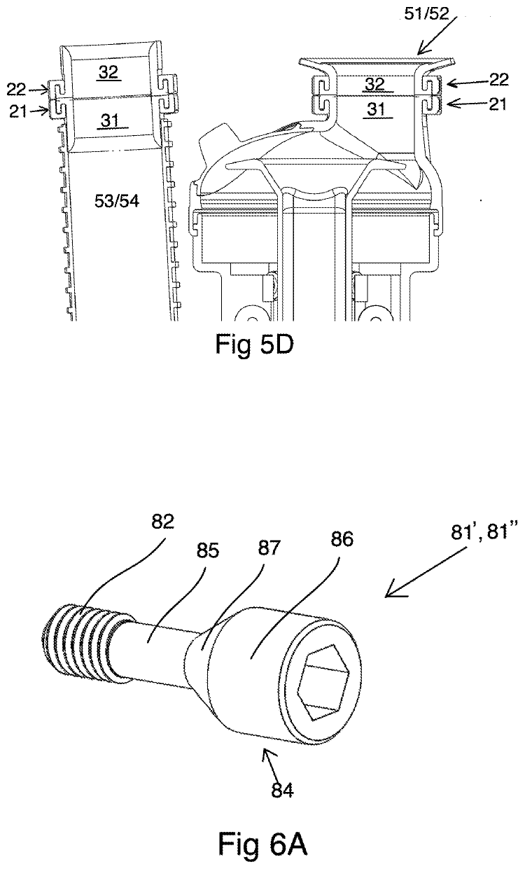

The second open end 37 of the first tubular connecting element 31 is arranged to be connected to either one of the inlet or the outlet channels of the TAH and is advantageously provided with a tubular element material having a length adapted to accommodate any strains that may appear in the connection between the vascular coupling device 10 and the TAH. The second end 37 may e.g. be reinforced with a metal wire to improve on the rigidity of the connection. For example, when the second end 37 of the first tubular connecting element 31 is connected to the inlet of the TAH, the second end 37 is advantageously shorter than when connected to the outlet channel (see e.g. FIG. 5D) and could be integrated into the material of the TAH.

The second open end 38 of the second tubular connecting element 32 is arranged to be connected to the vascular system of a patient and is advantageously provided with a vascular grafting material. The vascular grafting material must be blood compatible and is chosen from the group comprising any known commercially available vascular grafting materials such as e.g. polyethylene teraphthalate (Dacron), expanded polytetrafluoroethylene (ePTFE), Polyamide (nylon) and polyurethane. In one embodiment the vascular grafting material is polyethylene teraphthalate (Dacron).

However, it is also possible that the second open end 37 of the first tubular connecting element 31 is instead fitted with vascular grafting material, and the second open end 38 of the second tubular connecting element 32 is provided with the tubular element material to be connected to a TAH. Alternatively the second ends 37, 38 of both the first and second tubular connecting elements 31, 32 are provided with a vascular grafting material.

The first and second coupling elements 21, 22 are removably connected to each other into a locked configuration, or disconnected from each other into an unlocked configuration by means of a pair of locking structures 41, 42. The centerline A for the vascular coupling device 10 extends through the pair of locking structures 41, 42 which are arranged on the centerline A and opposite to each other on an outer perimeter of said vascular coupling device 10 (see FIG. 1D).

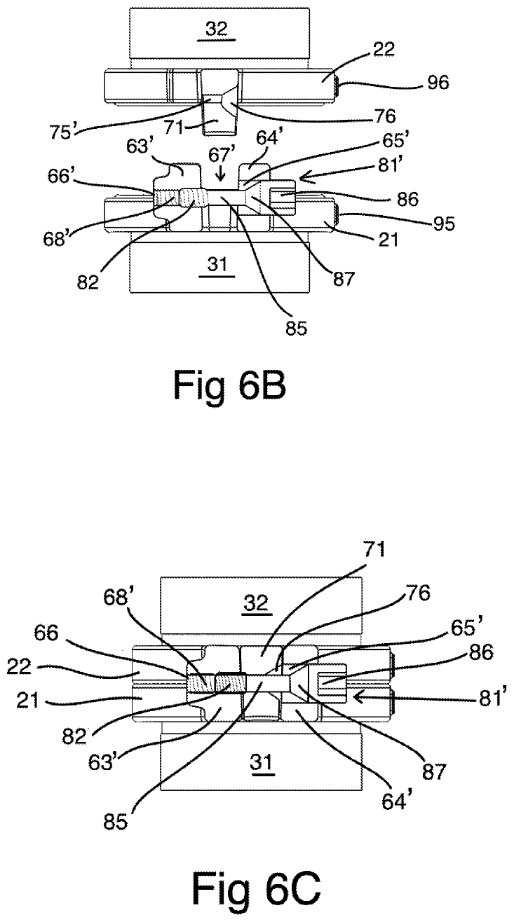

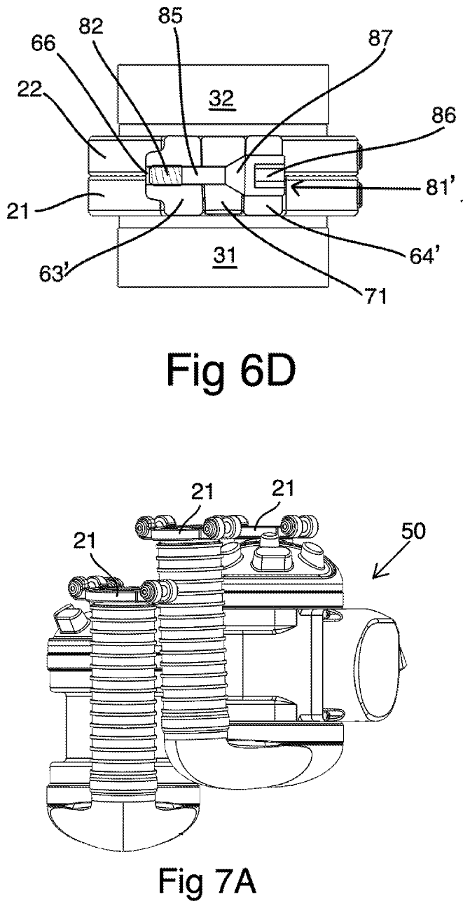

Each one of the locking structures 41, 42 comprises
a female locking part 61, 62 arranged on the first coupling element 21; and
a male locking part 71, 72 arranged on the second coupling element 22; and
a locking element 81′, 81″ (see FIG. 6A) configured to join the female locking part 61, 62 with the male locking part 70, 71 into a locked configuration (see FIGS. 1A-D and 2A-B). In the locked configuration the first and second coupling elements 21, 22 are brought into contact such that the docking surfaces 26′, 26″ on the flanges 34′, 34″ provided on the first open ends 33′, 33″ of the first and second tubular connecting elements 31, 32 meet and form a leak-proof conduit through which blood may pass (see FIGS. 1D and 4C).

The vascular coupling device 10 comprises two locking structures 41, 42 (FIG. 1D) located opposite to each other on the centerline A of the vascular coupling device 10. Thus, each vascular coupling device 10 comprises a first and a second female locking part 61, 62 arranged opposite to each other on the centerline A of the first coupling element 21, a first and a second male coupling part 71, 72 arranged opposite to each other on the centerline A of the second coupling element 22, and a first and a second locking element 81′, 81″ configured to join the first female locking part 61 to the first male locking part 71, and the second female locking part 62 to the second male locking part 72 respectively (see FIGS. 1C-D).

The first and second female locking parts 61, 62 are located on the centerline A and on the outer perimeter of the first coupling element 21. Each female locking part 61, 62 comprises a first lug 63′, 63″ and a second lug, 64′, 64″, with a gap 67′, 67″ located between the first lug 63′, 63″ and second lug 64′, 64″. As pointed out above, the pair of locking structures 41, 42 is arranged opposite to each other on the centerline A of the vascular coupling device 10, and consequently the first and second female locking parts 61, 62 are also arranged opposite to each other on the centerline A. Accordingly, the centerline A runs through the gaps 67′, 67″ of the first coupling element 21 placing said first lugs 63′, 63″ and second lugs 64′, 64″ on opposite sides B and C of said centerline A (see FIG. 2B).

The first lug 63′, 63″ has a first bore 65′, 65″ (located on side B of the centerline A), and the second lug 64′, 64″ has a second bore 66′, 66″ (located on side C of the centerline A), the second bore 66′, 66″ being provided with a screw thread on the inside wall 68′, 68″. The first bore 65′, 65″ has a first diameter which is larger than the diameter of the second bore 66′, 66″ (see FIGS. 2A and 6B).

The first and second male locking parts 71, 72 are arranged on the centerline A and opposite to each other on the outer perimeter of the second coupling element 22 only. The male locking parts 71, 72 are configured to be received into the gaps 67′, 67″ between the first lugs 63′, 63″ and second lugs, 64′, 64″ of the female locking parts 61, 62 (see FIGS. 1D and 2A-B).

The female and male locking parts 61, 62, 71, 72 respectively are connected together into a locked configuration by means of a locking element 81′, 81″. The locking element 81′, 81″ has a first end provided with an outer screw thread 83, a second end provided with a screw head 84, and a mid-section 85 arranged in between the first and second ends. As can be seen in FIG. 6A, the screw head 84 has two ends, a first end which has a cylindrical shape 86 and a second end which tapers from the cylindrical part into a cone shape 87 towards the mid-section 85 of the locking element 81′, 81″. The reason for this shape will be explained below.

The cylindrical part 86 on the first end of the screw head 84 has a diameter which matches the first diameter of the first bore 65′, 65″ of the female locking part 61, 62, and the screw threaded end 83 has a diameter that matches the diameter of the second bore 66′, 66″. The locking element 81′, 81″ is configured to be received into the first and second bores 65′, 65″, 66′, 66″ of the female locking part 61, 62 (see FIG. 6B).

The locking structure 41, 42 as described herein may show some similarities to a clevis joint but with some important differences which will be pointed out. The female locking part 61, 62 may have the shape and take the function of a clevis bracket. The male locking part 71, 72 may have the shape and take the function of a clevis eye, and the locking element 81 may have the shape and take the function of a clevis pin.

The locking elements 81′, 81″ are configured to enter with their first end 83′, 83″ (screw threaded end) through the first bore 65′, 65″ perpendicular to and from the same side B of the centerline A. Thereafter the first screw threaded ends 83′, 83″ cross the centerline A and are received into the second bores 66′, 66″ located on the opposite side C of the centerline A, while the second ends 84′, 84″ (i.e. the screw head) remain in the first bores 65′, 65″. This can be seen in FIGS. 1D, 2A-B and 6B-D wherein it is shown that both of the screw heads 84′, 84″ of the locking elements 81′, 81″ are arranged in the first bores 65′, 65″ of both of the female locking parts 61, 62 and on the same side B of the centerline A. The mid-sections 85′, 85″ are arranged in the gaps 67′, 67″ between the first and second lugs 63′, 63″, 64′, 64″, reaching across the centerline A, and the screw threaded ends 84′, 84″ are arranged inside the second bores 66′, 66″ on the opposite side C of the centerline A.

Each one of the male locking parts 71, 72 comprises an eye (or bore) 73, 74 provided with an open cleft 75′, 75″. The open clefts 75′, 75″ have a width corresponding to the diameters of the mid-sections 85′, 85″ of the locking elements 81′, 81″ with their openings facing towards the coupling side 25″ of the second coupling element 22 (see FIGS. 2A and 4A-C).

The mid-sections 85′, 85″ of the locking elements 81′, 81″ are configured to be received into the open clefts 75′, 75″ provided on the eyes 73, 74 of the male locking parts 71, 72 when the male locking parts 71 72 are arranged in the gaps 67′, 67″ between the first and second lugs 63′, 63″, 64′, 64″ of the female locking parts 61, 62 (see FIGS. 6B-C).

Each eye 73, 74 is provided with a first and second recess 76, 77 arranged on one side of the eye 73, 74 only. As seen in FIG. 1B both of the first and second recesses 76, 77 are located on side B of the centerline A. The first and second recesses 76, 77 are configured to receive the cone shaped ends 87′, 87″ of the locking element screw heads 84′, 84″. Both of the first and second recesses 76, 77 are arranged on the eyes 73, 74 such that they face in the same direction and away from the centerline A.

One advantageous feature of the vascular coupling device 10 described herein, is that the operator can be assured that the vascular coupling device 10 always is fitted in a correct way. This is extremely important since an incorrect assembly of the vascular coupling device 10 can have devastating consequences. In order to avoid problems of accidental incorrect fitting, the vascular coupling device 10 disclosed herein is provided with a fail-safe arrangement. This fail-safe arrangement comprises first and second cut-in portions 91, 92 arranged on the first coupling element 21 that cooperate with first and second projecting elements 93, 94 arranged on the second coupling element 22.

The first and second cut-in portions 91, 92 are arranged on an outer perimeter of the first coupling element 21. In the FIGS. 2A-B the cut-in portions 91, 92 form a part of the female locking part 61, 62 on the first coupling element 21, but the exact location on the first coupling element 21 is not important as long as both the first and the second cut-in portions 91, 92 are located on the same side of the centerline A, i.e. either both are located on side B or on side C. In the embodiments show in the figures, the first and second cut-in portions 91, 92 are arranged on the coupling side of the second lug 64′, 64″, i.e. on side C of the centerline A (see FIGS. 1D and 2A-B).

The first and second projecting elements 93, 94 are arranged on an outer perimeter of the second coupling element 22. In the FIGS. 2A-B the projecting elements 93, 94 are located next to the male locking part 71, 72 but the exact location on the outer perimeter is not important as long as both the first and the second projecting elements 93, 94 are located on the same side of the centerline A and arranged to cooperate with the first and second cut-in portions 91, 92 on the first coupling element 21, when the vascular coupling device 10 is in the locked configuration. In the embodiments shown in the figures, the first and second projecting elements 93, 94 are arranged on the C side of the centerline A, and opposite to the first and second recesses 76, 77 provided around the eyes 73, 74 (see FIG. 1B).

In order to function as a fail-safe arrangement, an erroneous assembly of the coupling elements 21, 22 must be prevented. The first and second projecting elements 93, 94 are configured to be received into the first and second cut-in portions 91, 92 when the vascular coupling device 10 is in a locked configuration. Consequently the first and second cut-in portions 91, 92 on the first coupling element 21 must be located such that they will mate with the first and second blocking elements 93, 94 on the second blocking element 22 when the vascular device is in the locked configuration.

This mirror image configuration of the fail-safe arrangement prevents any incorrect assembly of the vascular coupling device 10. It is therefore impossible to assemble the first and second locking structures 41, 42 in an incorrect manner since trying to fit the first and second locking elements 21, 22 together when one of them is turned the wrong way is prevented by the projecting elements 93, 94 not being able to be fitted into the cut-in portions 91, 92. When the cut-in portions 91, 92 are arranged on the second lugs 64′, 64″ as seen in the FIGS. 2A-B any attempt to assemble an incorrectly positioned coupling element 21, 22, e.g. the first coupling element 21 to the second coupling element 22 will be prevented when the projecting elements 93, 94 arranged on the second coupling element 22 end up opposite to the first lugs 63′, 63″ on the first coupling element 21 (i.e. the lugs without the cut-in portions). It is therefore impossible to assemble the first and second coupling elements 21, 22 together the wrong way.

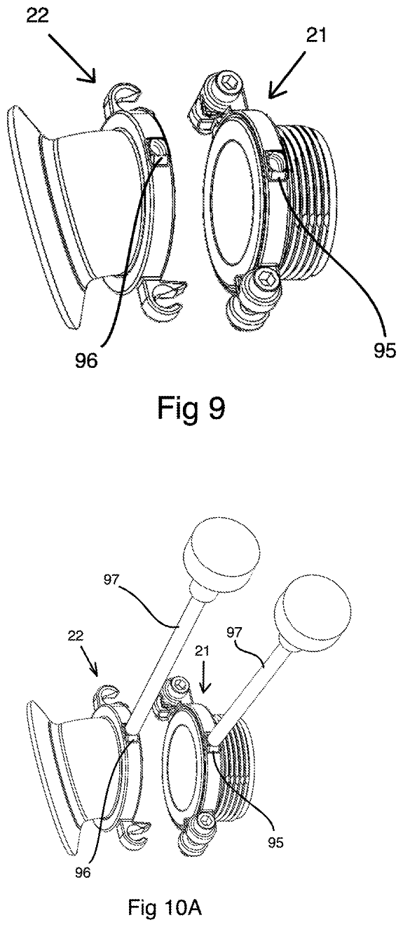

In order to facilitate the handling of the vascular coupling device 10 during implantation of the vascular coupling device to the vascular system of the patient, the vascular coupling device 10 is provided with first and second receiving means 95, 96, advantageously provided with inner screw threads (see FIG. 9). The first receiving means 95 is located on the first coupling element 21 and the second receiving means 96 is located on the second coupling element 22, both first and second receiving means 95, 96 are located on the same side of the center line A. As can be seen in FIGS. 10, 2B and 9A, the first and second receiving means 95, 96 are located on the B side of the centerline A. However, the first and second receiving means 95, 96 may of course be located on the opposite side C instead.

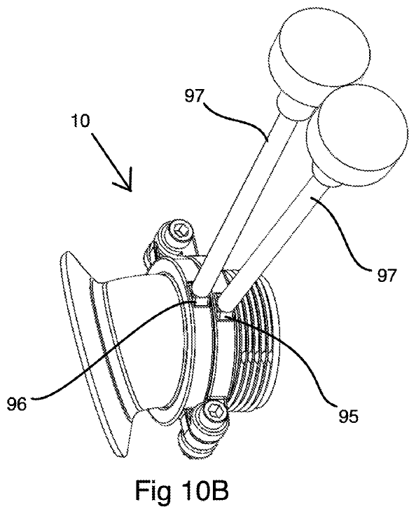

Each one of the first and second receiving means 95, 96 are configured to receive a holding means 97 (see FIGS. 10A and 10B), configured to be connected thereto and used for holding and manipulating the vascular coupling device 10 during the implantation process. The holding means 97 is advantageously provided with an outer screw thread adapted to mesh with the inner screw thread provided in the first and second receiving means 95, 96.

A method of fitting a TAH to a vascular system of a patient by means of the vascular coupling device 10 as disclosed herein will now be described in more detail. An advantageous feature of the vascular coupling device 10 is that the operator is able to connect and/or disconnect the vascular system of a patient to a TAH or other type of heart pump device quickly without having to perform many time consuming steps. It is an advantage that most of the parts of the vascular coupling device 10 can be prepared and fitted beforehand in order for the connecting/disconnecting operation to take a minimum amount of time.

The first and second tubular connecting elements 31, 32 are fitted into the first and second coupling elements 21, 22 respectively such that the downwardly/upwardly turned lips 35′, 35″ are resting inside the grooves 29′, 29″ on the inner parameters of the central openings 27′, 27″, and the second open ends 37, 38 are protruding through the central openings 27′, 27″ towards the external sides of each coupling element 21, 22 (see FIGS. 1B and 4B). This step is advantageously performed before surgery has started.

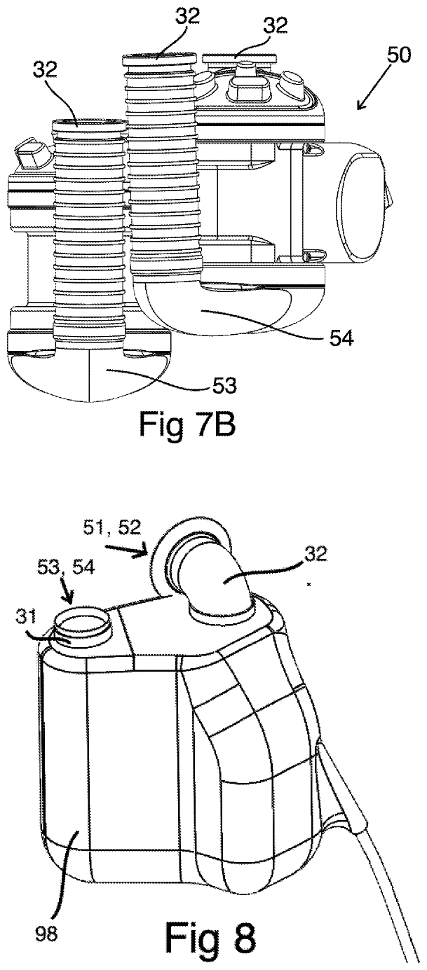

The second open end 37 of the first tubular connecting element 31 protruding from the central opening 27′ of the first coupling element 21 is advantageously provided with a rigid tubular element reinforced with a metal wire which is used for connecting the vascular coupling device 10 to an inlet 51, 52 or outlet 53, 54 of a TAH 50. This connection may be prepared in advance of the surgery such that the rigid tubular element reinforced with a metal wire is connected to the inlet/outlet of the TAH and the first end 33′ of the first tubular connecting element 31 provided with the flange 34′ and the downwardly turned lip 35′ is mounted in the groove 27′ on the first coupling element 21. Advantageously it is the first coupling element 21 comprising the female locking parts 61, 62 that are arranged on the inlets 51, 52/outlets 53, 54 of the TAH 50 in advance (see FIG. 7A). However, it is possible that under some circumstances also the second coupling element 22 comprising the male coupling parts 71, 72 may be fitted to the inlet 51, 52/outlet 53, 54 of the TAH 50 already at the start of the procedure. As a TAH 50 normally has two inlet channels 51, 52 and two outlet channels 53, 54. All four channels 51, 52, 53, 54 may be fitted with at least a tubular connecting element 31 (see FIG. 7B) but optionally also with the coupling element 21, 22 of the vascular coupling device 10 before surgery has started (see FIG. 7A).

In order to transfer the vascular coupling device 10 into a locked configuration, both of the locking elements 81′, 81″ are entered (from the same side, i.e. side B of the center line A) through the first bores 65′, 65″ of the female locking parts 61, 62 such that the outer screw threads 82′, 82″ of the locking elements 81′, 81″ are received into and partly joined with the inner screw threads 68′, 68″ provided inside the second bores 66′, 66″. The screw heads 83′, 83″ of the locking elements 81′, 81″ are received into and rest inside the first bores 65′, 65″. This step may also be prepared before starting surgery (see FIGS. 1B, 4B and 6B).

When the chest of the patient has been opened and the biological heart removed, the vascular grafting material provided on the on the second end 38 of the second tubular connecting element 32 may be connected to the arteries or veins, alternatively to the remaining parts of the natural atria of the natural heart. Advantageously, the second tubular connecting element 32 has already been fitted into the second coupling element 22 such that the upwardly turned lip 35″ is resting inside the groove 29″ along the inner parameter of the central opening 27″, and the second open end 38 with the vascular grafting material is protruding through the central opening 27″ towards the external side of the second coupling element 22. Consequently the male locking parts 71, 72 are arranged on the part of the vascular device 10 connected to the arteries/veins alternatively the remaining parts of atria.

In order to connect the vascular device 10 into a locked configuration, the first and second male locking parts 71, 72 are placed into the gaps 67′, 67″ arranged between the first and second lugs 63′, 63″, 64′, 64″ of the female locking parts 61, 62 such that the mid-sections 85′, 85″ of the locking elements 81′, 81″ are received into the open clefts 75′, 75″ of the eyes 73, 74 on the male locking elements 71, 72 (see FIGS. 1C, 4C and 6C). This will not be possible unless both of the first and second coupling elements 21, 22 are turned in the right direction. If one of the coupling elements 21, 22 are turned the wrong way, the first and second coupling elements 21, 22 will not assemble due to the fail-safe arrangement.

The rotational capability of the tubular connecting elements 31, 32 in relation to the coupling elements 21, 22 is a great advantage during the implantation process since if there is some strain between the vascular coupling device 10 and the veins/arteries of the patient, or between the vascular device and the TAH, the surgeon may then rotate one or both of the tubular connecting elements 31, 32 to optimize the fitting and release the strain or stress.

Thereafter, the ends of the locking element 81′, 81″ provided with screw threads 82′, 82″ are screwed all the way into the second bores 66, 66′, thereby causing the cone shaped part 87′, 87″ of the screw heads 84, 84′ to be received into the first and second recesses 76, 77 provided on the eyes 73, 74 of the male locking parts 71, 72 (see FIG. 6D). When the cone shaped part 87′, 87″ of the screw heads 84′, 84″ enter the first and second recesses 76, 77 of the eyes 73, 74, the first and second coupling elements 21, 22 are pulled together further into a firm locking configuration. Simultaneously the coupling surfaces 26′, 26″ on the flanges 34′, 34″ provided on the first open ends 33′, 33″ of the first and second tubular connecting elements 31, 32 meet and form a tight and leak-proof conduit through which blood may pass (see FIG. 4C).

Advantageously an encasing sac 98 is attached to the external surfaces 23′ of the coupling device 10 facing away from the TAH when the vascular coupling device 10 is connected to the inlet channels and outlet channels 53, 54 of the TAH (see FIG. 8). The encasing sac 98 receives and encloses the artificial heart pumps 50 to protect the pump from tissue ingrowth.

1. A vascular coupling device (10), comprising
a first and a second coupling element (21, 22) wherein each one of said first and second coupling elements (21, 22) has an external surface (23′, 23″) facing an external side, a coupling surface (25′, 25″) facing a coupling side, and a central opening (27′, 27″); and
a first and a second tubular connecting element (31, 32), wherein each one of said first and second tubular elements (31, 32) has a first open end (33′, 33″) and a second open end (37, 38), said first open ends (33′, 33″) being provided with flanges (34′, 34″);
each one of said first and second tubular connecting elements (31, 32) is arranged in a corresponding central opening (27′, 27″) of the first and second coupling elements (21, 22) respectively, and with said second open ends (37, 38) protruding through said central openings (27′, 27″) on said external side of each of said first and second coupling elements (21, 22);
said first and second coupling elements (21, 22) being removably connected to each other into a locked configuration, or disconnected from each other into an unlocked configuration by means of a first and second locking structure (41, 42), said first and second locking structures (41, 42) being arranged on a centerline A of the vascular coupling device (10), and opposite to each other on an outer perimeter of said vascular coupling device (10),
characterized in that said vascular device further comprises a fail-safe arrangement comprising first and second cut-in portions (91, 92) arranged on said first coupling element (21) configured to receive first and second projecting elements (93, 94) arranged on said second coupling element (22), said first and second cut-in portions (91, 92) and first and second projecting element (93, 94) being arranged on same side of said centerline A, thereby preventing erroneous connection of said first and second coupling elements (21, 22) to each other.

2. The vascular coupling device according to claim 1, wherein inner perimeters of said central openings (27′, 27″) are provided with grooves (29′, 29″) facing said coupling sides.

3. The vascular coupling device according to claim 1, wherein said flanges (34′, 34″) on said first open ends (33′, 33″) of each of said first and second tubular connecting elements (31, 32) are provided with downwardly/upwardly turned lips (35′, 35″).

4. The vascular coupling device according to claim 3, wherein said downwardly/upwardly turned lips (35′, 35″) provided on said flanges (34′, 34″) are resting in said grooves (29′, 29″) on the inner diameters of the central openings (27′, 27″).

5. The vascular coupling device according to claim 1, wherein each one of said first and second locking structures (41, 42) comprises
a female locking part (61, 62) arranged on said first coupling element (21); and
a male locking part (71, 72) arranged on said second coupling element (22); and
a locking element (81′, 81″) configured to join said female locking part (61, 62)
with said male locking part (71, 72) into a locked configuration, wherein said coupling surfaces (25′, 25″) of said first and second coupling elements (21, 22) are brought into contact such that said first open ends (33′, 33″) of said first and second tubular connecting elements (31, 32) are brought into a sealing connection

6. The vascular coupling device according to claim 5, wherein
each one of said female locking parts (61, 62) comprises a first and a second lug (63′, 63″, 64′, 64″) having a gap (67′, 67″) therebetween, said first and second lugs (63′, 63″, 64′, 64″) being arranged on opposite sides of said centerline A, said first lug (63′, 63″) having a first bore (65′, 65″), and said second lug (64′, 64″) having a second bore (66′, 66″) provided with an inner screw thread; and
each one of said male locking parts (71, 72) is arranged on the centerline A and is configured to be received into said gap (67′, 67″) between said first and second lugs (63′, 63″, 64′, 64″) of said female locking parts (61, 62); and
each one of said locking elements (81′, 81″) has a first end provided with an outer screw thread (82′,82″), a second end provided with a cone shaped head (87′, 87″), and a mid-section (85′, 85″) arranged between said first and second ends, said locking elements (81′, 81″) are configured to be received into said first and second bores (65′, 65″, 66′, 66″) of said female locking part (61, 62).

7. The vascular coupling device according to claim 6, wherein both of said locking elements (81′, 81″) are configured to be entered into said first bores (65′, 65″) from a first side and perpendicular to said centerline A, cross said centerline A and thereafter be received into said second bores (66′, 66″) provided with inner screw threads and arranged on a second side of said centerline A opposite to said first side.

8. The vascular coupling device according to claim 1, wherein each one of said male locking parts (71, 72) comprises an eye (73, 74) provided with an open cleft (75′, 75″) facing said coupling side of said second coupling element (22).

9. The vascular coupling device according to claim 8, wherein said mid-sections (85′, 85″) of said locking elements (81′, 81″) are configured to be received into said open clefts (75′, 75″) provided on said eyes (73, 74) of said male locking parts (71, 72) when said male locking parts (71, 72) are arranged in said gaps (67′, 67″) between said first and second lugs (63′, 63″, 64′, 64″) of said female locking parts (61, 62).

10. The vascular coupling device according to claim 8, wherein first and second recesses (76, 77) are arranged around inner perimeters of said eyes (73, 74) and on same side of said centerline A of said male locking parts (71, 72), said first and second recesses (76, 77) being configured to receive said cone-shaped heads (87′, 87″) of said locking elements (81′, 81″).

11. The vascular coupling device according to claim 8, wherein said vascular (10) device is in a locked configuration when said outer screw threads (82′, 82″) of said locking elements (81′, 81″) are received into and joined with said inner screw threads provided in said second bores (66′, 66″) of said female locking parts (61, 62), and said cone-shaped heads (87′, 87″) of said locking elements (81′, 81″) are received into said recesses (76, 77) provided on said eyes (73, 74) arranged on said male locking parts (71, 72).

12. The vascular coupling device according to claim 1, wherein said first and a second projecting element (93, 94) are arranged on an outer perimeter of said second coupling element (22)

13. The vascular coupling device according to claim 1, wherein said first and second projecting elements (93, 94) and said recesses (76, 77) are arranged on opposite sides of said centerline A of said second coupling element (22).

14. The vascular coupling device according to claim 1, wherein said first and a second cut-in portions (91, 92) are arranged on an outer perimeter of said first coupling element (21).

15. The vascular coupling device according to claim 1, wherein an encasing sac (98) is attached to the external surface (23′, 23″) of the coupling device and receives and encloses the artificial heart pump 50 to protect the pump from tissue ingrowth.

16. The vascular coupling device according to claim 1, wherein a first and second receiving means (95, 96) are arranged on the first and second coupling elements (21, 22) respectively, both first and second receiving means (95, 96) being arranged on the same side of the center line A and are adapted to facilitate manipulation of said vascular coupling device (10) during implantation.

17. A total artificial heart (TAH) (50) comprising a first and a second inlet channel (51, 52) and a first and a second outlet channel (53, 54), wherein one or more of the first and second inlet and outlet channels (51, 52, 53, 54) are connected to a vascular coupling device (10) according to claim 1.
